(19) United States
(12) Patent Application Publication
(10) Pub. No.: US 2005/0004542 A1
(43) Pub. Date: Jan. 6, 2005

(54) Absorbent pad system and method of manufacture

(76) Inventor: Charles A. Bakkila, Northville, MI (US)

Correspondence Address: CHARLES C. VALAUSKAS;BANIAK PINE & GANNON, Suite 1200, 150 N. Wacker Drive, Chicago, IL, 60606, US

(21) Appl. No.: 10/884,490

(22) Filed: Jul. 2, 2004

Related U.S. Application Data
(60) Provisional application No. 60/484,970, filed on Jul. 3, 2003.

(51) Int. Cl. A61F 13/15; A61F 13/20

(52) U.S. Cl. 604/367

(57) ABSTRACT

The invention is an absorbent pad system and method of manufacturing same. A preferred embodiment of the invention includes a non-woven absorbent core fabric material coated on one flat planar surface with a polyester film interposed between two distinctly separate layers of acrylic adhesive, and optionally with a wide-mesh material adhesively affixed to the other flat planar surface to obtain an absorbent device that is conveniently adaptable to a large variety of usages. A method of manufacture is described.

### CROSS-REFERENCE TO RELATED APPLICATIONS

This application claims the benefit of U.S. Provisional Application No. 60/484,970, filed Jul. 3, 2003.

### FIELD OF THE INVENTION

The present invention relates to an improved type of replaceable moisture and liquid absorbent mat or pad, and to a method for its manufacture. In particular, one embodiment of the present invention provides a composite non-woven fabric core material that has substantial capacity for absorbing and sequestering liquid fluids from across a semi-permeable thin film, to a defined limit of saturation. A moisture-resistant barrier including an acrylic adhesive is preferably used over the non-woven fabric material to secure the composite absorbent pad of the present invention in place during the period of its use.

### BACKGROUND OF THE INVENTION

Because there are a myriad number of manufactured products made from absorbent fabric materials, it is envisioned that improvements obtained in their formulation and construction as provided by the present invention may find a wide range of applicable uses. At least several such uses might be mentioned, without intending to imply any necessary limitation or exclusion thereby. As a manufactured fabric, the article of the present invention is sufficiently comfortable for wearing with other items of clothing, so that it could be incorporated for use with absorbent incontinence pads, with brassiere liners worn by lactating women, or with hat bands or athletic head bands worn to provide a barrier against unwanted effluence of perspiration. Combined with other types of manufactured materials, the present invention may be useful to improve the functional effectiveness of decorative coasters for beverage glasses or serving dishes, or vases containing indoor plants or flowers. An absorbent material of the present invention could also be used with replaceable floor covering products to provide a protective absorptive floor mat, for areas around urinals or pet litters and commodes, in residential, commercial, institutional, airplane, RVs and the like. The absorbent fabric material of the present invention might also be used as a disposable absorbent safety mat, for reducing the extent of incidental exposure to hazardous or environmentally toxic substances.

It is therefore a primary object of the present invention to provide an absorbent fabric material that is adaptable to a wide range of potential usages, wherein the fabric material has substantial absorbent capacity.

It is another object of the present invention to provide an absorbent fabric component that may be firmly secured to a variety of substrates, but that is also conveniently detachable for replacement with another absorbent pad device, as may ordinarily be required or desirable during normal use. The absorbent core can be impregnated with disinfectants, deodorizers, and/or antibacterial agents as needed for the various applications.

Another object of the present invention is to provide an absorbent pad that has a moisture-resistant adhesive coating on one flat planar surface, whereby the absorbent pad device of the invention may be held securely in place during ordinary use, even when wet.

Yet another object of the present invention is to provide an absorbent fabric material core that contains a mesh or net material on another flat planar surface, attached securely by waterproof and heat activated adhesive to the absorbent core of multilayered non-woven fabric material.

There is a demand for an absorbent fabric material that combines superior performance and low manufacturing and maintenance costs. The present invention satisfies the demand.

### SUMMARY OF THE INVENTION

These and other objects of the invention are provided, in one embodiment, by an absorbent core and barrier arrangement. The absorbent core preferably includes a non-woven absorbent core fabric material coated on at least one flat planar surface with the barrier. The barrier may include a polyester film interposed between two distinctly separate layers of acrylic adhesive, for example, and optionally with a wide-mesh material adhesively affixed to the other flat planar surface, thus to obtain an absorbent device that is conveniently adaptable to a large variety of usages. In accordance with the present invention, the absorbent core material draws and stores fluid liquids, to a certain limited saturation capacity. Liquid fluids that transfer into the absorbent core from across a thin laminated film are substantially prevented from leaking away, both to the extent that the absorbent core is not fully saturated, and to the extent that the barrier of the thin laminated film may prevent exudation from the absorbent core. The surface of the outermost acrylic adhesive layer may be protected by a flexible release sheet made from paper or other similar material, which is conveniently removed just prior to use, to expose the adhesive surface of the absorbent pad device beneath.

These and other objectives and advantages of the present invention will become evident from the following drawings and the accompanying detailed descriptions of them. The product can be cut, formed or provided, for example, in various sizes and shapes to accommodate various uses. The product can contain a type of litmus material, around the perimeter, for example, which would change color when it comes in contact with liquids thus indicating a time to change the product.

An advantage of a product according to the present invention is that it is easy to apply to various surfaces such as, for example, hats, helmets, band hats, bras, clothing, floors, counters, table surfaces and so on. The product according to the present invention may include use in trash can liners, alternate hat band shapes including curves and shapes for various usage, an absorbent transport pad liner for preservation of specimens, and shoe insole liners.

The polyester barrier film is pliable and flexible so that it will conform to surfaces, which are uneven, or of different shapes. The film is thin, in part, to the fact that the material layers are bonded, preferably thermally. The present invention may include a releasable adhesive to secure it in place. The present invention provides the capability to encapsulate absorbed liquids. The present invention can be cut or otherwise formed into application-specific shapes without significant sacrifice of its properties.

### BRIEF DESCRIPTION OF THE DRAWINGS

While the claims concluding the specification particularly point out and distinctly claim the precise subject matter regarded as the sum and substance of the present invention, its construction and composition may be best understood from the following detailed description taken in connection with the accompanying drawings, in which the thickness of some of the component layers have been exaggerated for clarity of illustration, and in which:

FIG. 1 shows a side elevation view of a multilayered, non-woven absorbent core;

FIG. 2 shows the same absorbent core of FIG. 1, as a unitary composite after thermal bonding;

FIG. 3 shows a side view of polyester film with two adjacent layers of adhesive, and with a protective release sheet covering one of adhesive layers;

FIG. 4 shows a side view of the combined layers shown in FIG. 3;

FIG. 5 shows an edge of mesh material that may optionally be combined with the absorbent core material;

FIG. 6 shows the absorbent core illustrated in FIG. 1 and the several added layers of material shown in FIG. 3, combined; and

FIG. 7 shows the same layers depicted in FIG. 6, combined with the optional mesh material.

### DETAILED DESCRIPTION OF A PRESENTLY PREFERRED EMBODIMENT

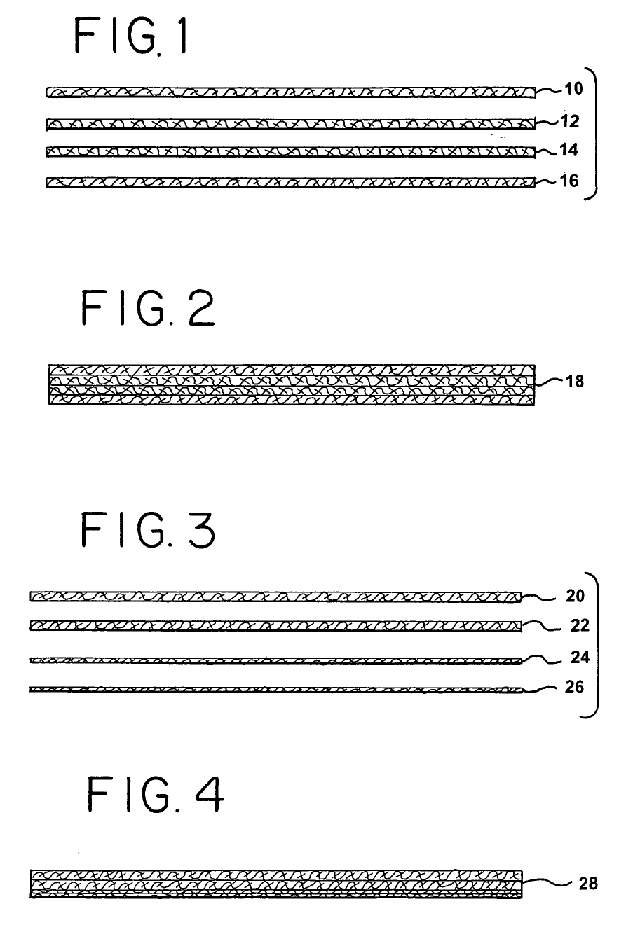

The device of the present invention is a pad 8 (see FIG. 6) that includes an absorbent core 18 (see FIG. 2). The absorbent core 18 is preferably made of non-woven fabric material. The absorbent core 18 preferably is formed from a plurality of absorbent layers 10, 12, 14, and 16 (see FIG. 1). FIG. 1 shows one preferred embodiment of the absorbent core 18 formed from four layers comprising two layers, 10, 12 and 14, 16 of similar or identical half-thicknesses of material, each including two air-laid panels, similar to the arrangement shown in U.S. Pat. No. 3,952,124, with the two layers 12, 14 of half thicknesses joined back-to-back. The two outer-most layers 10, 16 of the resulting non-woven composite fabric may be made from 80% synthetic bonding fibers and 20% fluff pulp. Added bonding fibers may be added to provide a strong, smooth surface having a comfortable and non-abrasive texture. The fluff pulp provides absorbency as well as wicking action to absorb liquid. The contiguous inner layers 12, 14 of the non-woven composite are preferably made from about 50% super absorbent fibers, fiber and pulp. Similar to the manufacturing process described in U.S. Pat. No 4,186,165, the combined layers 10, 12, 14, and 16 may be compacted through a roller, to provide a densified absorbent structure 10. The structure 10, 12, 14, and 16 is then passed through a thermal oven in which heated air (preferably at about 145° C.) is blown through the structure 10, thereby bonding all four layers 10, 12, 14, 16 together to form a unitary absorbent core 18, similar to the process described in U.S. Pat. Nos. 4,103,062 and 6,194,630, as illustrated in the diagram of FIG. 2. The contents of the fluff pulp and the super-absorbent layers 12, 14, 16, and 18 are preferably thermally bonded together to obtain absorbent core 18 thereby obviating the need for adhesives.

In an alternate process, the plurality of layers 12, 14, 16, and 18 may be organized as above (FIG. 1) and the absorbent core 18 is formed by embossing. The process of embossing provides benefits including strengthened structure of absorbent core 18. Thermally bonding the embossing pattern provides additional strengthening providing a core 18. Of course, it will be understood that any embossing pattern may be used. As is known in the art, the embossing is accomplished through use of male and female dies.

In one preferred embodiment, the absorbent core 18 fabric produced by the above noted compacting and/or embossing process comprises approximately 62% fluff pulp, 12% bonding fiber, and 26% super-absorbent polymer. The density of the composite absorbent core material may be about 200 gsm (equivalent to about 0.07 g/ccm) with a median thickness of about 3.27 mm. The dry tensile strength of the absorbent core may be in the range of 3356 g/50 mm. The absorbent volume capacity of the non-woven fabric can be at least 5.4 g/SQI per min for distilled water and 4.2 g/SQI per min for normal saline. The wicking rate for the composite material is in the range of 38.3 mm diameter/5 ml/min. Non-woven air-laid fabrics having such characteristics may be commercially available from several sources. Concert Fabrication Ltée, a manufacturing concern located in Gatineau, Quebec, supplies a thermal bonded absorbent core fabric designated as DT200.103.0001, with the same or approximately similar characteristics.

As discussed in the disclosure of U.S. Pat. No. 5,021,050 and European Patent No. 122,042, absorbent composites having an outer cellulosic layer and an inner region of super-absorbent material are known to have improved characteristics for rapid wicking action and an improved capacity for total volume of liquid absorbed. In such types of non-woven absorbent fabric, liquid that is drawn into outermost layers may then be transferred to more interior layered regions. The super-absorbent material thermally bonded to the more interior regions of the composite absorbs liquid more readily as compared to the outer layered regions, thus encapsulating the liquid within the product and away from the outer surface layers. As a result, the outermost layered regions and surfaces tend to remain dry.

In a separate manufacturing process, the absorbent core 18 (FIG. 2) is preferably combined with an adhesive laminated barrier material 28 (FIG. 4). The laminated barrier material 28 may be formed of three layers of material, a thin film of polyester enclosed within adjacent outer layers, each outer layer formed of a distinct type of acrylic adhesive. The adhesive laminate material may be combined with the non-woven absorbent fabric core by means that are well known in the art, such as by using a continuous roller method of the type described in U.S. Pat. Nos. 5,064,492 and 6,127,595. FIG. 3 shows one preferred embodiment of a laminated barrier material 28 (see FIG. 4) that includes a laminated structure 20, 22, 24, and 26. More specifically, FIG. 3 shows a first layer of the adhesive laminate 20, consisting essentially of an acrylic adhesive 20 that binds in contiguity with the absorbent core 18 (FIG. 2) made from a waterproof acrylic designed to maintain a permanent bond in an aqueous environment. The first adhesive laminate layer 20 is preferably formed in adjacent contiguity with a thin polyester film 22. Polyester film layer 22 can act both as the carrier layer for the first adhesive and as a carrier layer for a second type of acrylic adhesive 24 and also as a moisture barrier. The second adhesive 24 is designed to bond in the presence of moisture, and additionally may be cleanly removed from an underlying substrate, upon completion of product use. A type of adhesive laminate containing three such layers 20, 22, and 24 combined together, forming a roll product or thin sheet of a composite pressure sensitive adhesive, may be commercially available from Adhesives Research, Inc. of Glen Rock, Pa., as marketed under the brand name ARCLAD® and with a product code number designated IS-804928. This composite product combines a clear polyester film carrier with two distinctly separate layers of high performance acrylic adhesives, one adhesive designated by the numeric code AS-112, and a second adhesive designated by numeric code AS-46. As shown in FIG. 3, a protective layer 26 may include a release paper material that peels away from the outermost acrylic adhesive, to prevent exposure of the underlying adhesive layers until just prior to use, substantially as also described in U.S. Pat. No. 4,023,570, and as illustrated in the diagram of FIG. 6, showing an absorbent core 18 bonded to the laminated barrier material 28 to produce an embodiment of one product of the present invention. The protective layer 26 may include or consist of 84 pound poly-coated Kraft Paper or an equivalent thereof.

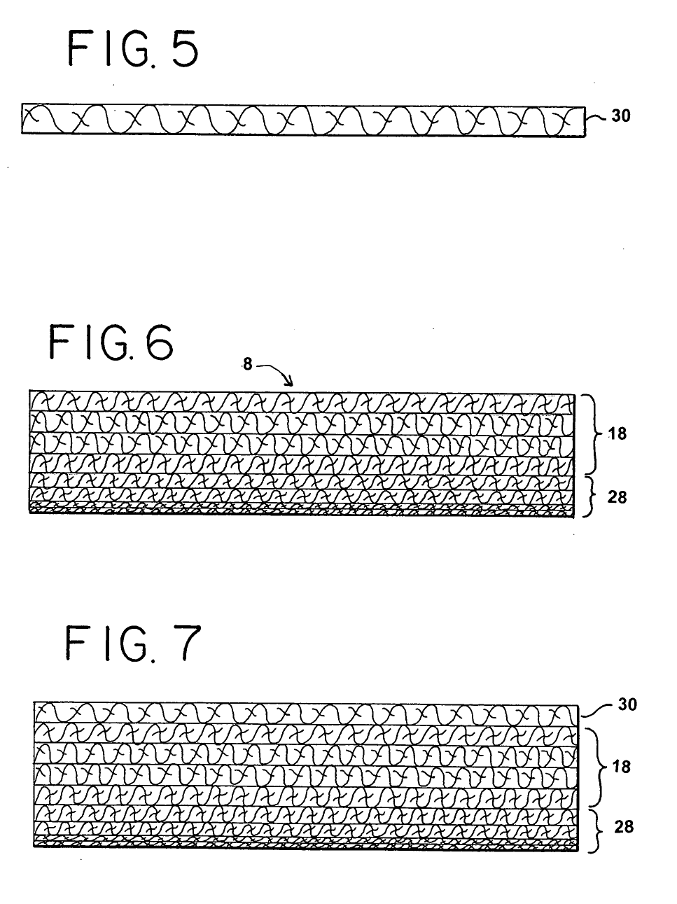

As illustrated in the diagram of FIG. 5, the device of the present invention may optionally also include a layer consisting of a wide-mesh material 30 providing a skid-resistant surface opposite the layers of acrylic adhesive and preferably bonded to the obverse surface by using a heat-activatable type of adhesive. Preferable types of wide-mesh weave may be made from a 4×4 wave of synthetic yarn having a diameter of approximately 0.02 to 0.03 inches, or from an 8×8 weave of synthetic yarn having a diameter of 0.1 to 0.2 inches. A type of wide-mesh material and a heat activated adhesive designated as HA-24 is currently commercially available from Adhesives Research, Inc. As illustrated in the diagram of FIG. 7, a device of the present invention may be assembled from mesh material 30 and laminated barrier material 28 with absorbent core 18 positioned between mesh material and laminated barrier material.

The present invention is usable in a number of applications. For example, the pad may be wetted with water and used in germinating seeds, e.g., for testing purposes, indoors or outside. Additionally, the pad may be used in a Petri dish or the like as a growth surface/medium for bacteria or similar organisms after being wetted and in the alternate, provided with various growth sustaining materials.

While the apparatus and method herein disclosed forms a preferred embodiment of this invention, this invention is not limited to that specific apparatus and method, and changes can be made therein without departing from the scope of this invention, which is defined in the appended claims.

1. An absorbent adhesive pad comprising:
an absorbent core formed of a combined total of about 62 percent fluff pulp, 12 percent bonding fiber, and 26 percent super-absorbent polymer, said absorbent core including a first outer layer of a mixture of about 20 percent fluff pulp and about 80 percent bonding fiber; one or more inner layer bonded to said first outer layer; and a second outer layer of a mixture of about 20 percent fluff pulp and about 80 percent bonding fibers bonded to said one or more inner layer; and
an adhesive laminate applied to an outer surface of at least one of said first outer layer of said absorbent core and an outer surface of said second outer layer of said absorbent core, said adhesive laminate including a polyester film positioned between a first acrylic adhesive layer and a second acrylic adhesive layer.

2. An absorbent adhesive pad comprising:
an absorbent core including a first outer layer of a mixture of about 20 percent fluff pulp and about 80 percent bonding fiber; one or more inner layer bonded to said first outer layer; and a second outer layer of a mixture of about 20 percent fluff pulp and about 80 percent bonding fibers bonded to said one or more inner layer; and
an adhesive laminate applied to an outer surface of at least one of said first outer layer of said absorbent core and an outer surface of said second outer layer of said absorbent core, said adhesive laminate including a polyester film positioned between a first acrylic adhesive layer and a second acrylic adhesive layer.

3. The adsorbent adhesive pad of claim 2, wherein said adhesive laminate is applied to both of said outer surface of said first outer layer of said absorbent core and said outer surface of said second outer layer of said absorbent core.

4. The absorbent adhesive pad of claim 2, wherein said inner layer of said absorbent core includes non-woven fabric material.

5. The absorbent adhesive pad of claim 4, wherein said inner layer of said absorbent core is formed of two half-thicknesses of said non-woven fabric material.

6. The absorbent adhesive pad of claim 4, wherein said non-woven fabric material includes at least 50 percent super-absorbent fibers.

7. The absorbent adhesive pad of claim 6, wherein said inner layer, said first outer layer and said second outer layer are thermally bonded together.

8. The absorbent adhesive pad of claim 6, wherein said inner layer, said first outer layer and said second outer layer are embossed.

9. The absorbent adhesive pad of claim 2, wherein said first acrylic adhesive layer and said second acrylic adhesive layer are formed of a material which provides moisture barrier properties.

10. The absorbent adhesive pad of claim 2, wherein said first acrylic adhesive layer is applied to said second outer layer of said absorbent core.

11. The absorbent adhesive pad of claim 10, further comprising a protective layer applied to said second acrylic adhesive layer.

12. The absorbent adhesive pad of claim 12, wherein said protective layer is Kraft paper.

13. The absorbent adhesive pad of claim 2, further comprising a layer of wide-mesh material applied to said absorbent core opposite said adhesive laminate.

14. The absorbent adhesive pad of claim 13, wherein said layer of wide-mesh material is bonded to said absorbent core by a heat-activatable adhesive.

15. A method of manufacturing an absorbent pad, comprising:
combining about 20 percent fluff pulp and about 80 percent bonding fiber to form an absorbent mixture;
forming the absorbent mixture into a first outer layer;
applying one or more inner layer of non-woven fabric material to the first outer layer;
organizing the absorbent mixture to form a second outer layer;
applying the second outer layer to the one or more inner layer to form an absorbent core; and
laminating an adhesive to an outer first surface of at least one of the first outer layer of the absorbent core and an outer second surface of the second outer layer of the absorbent core to form an adhesive laminate, the adhesive laminate including a polyester film positioned between a first acrylic adhesive layer and a second acrylic adhesive layer.

16. The method of claim 15, further including the step of compacting the absorbent core through a roller.

17. The method of claim 16, further including passing the absorbent core through a thermal oven to bond the first outer layer, inner layer and second outer layer into a unitary composite fabric material.

18. The method of claim 15, further including embossing the absorbent core after being formed.
